(19) United States
(12) Patent Application Publication
(10) Pub. No.: US 2014/0377371 A1
(43) Pub. Date: Dec. 25, 2014

(54) COMPOSITION FOR TREATMENT OF URINARY INCONTINENCE AND PREPARATION PROCESS THEREOF

(71) Applicant: Uchechi L. Eze, Springs, TX (US)

(72) Inventor: Uchechi L. Eze, Springs, TX (US)

(21) Appl. No.: 13/924,287

(22) Filed: Jun. 21, 2013

(51) Int. Cl. A61K 36/28; A61K 35/64

(52) U.S. Cl. CPC A61K 36/28; A61K 35/644
USPC 424/537

(57) ABSTRACT

A composition for Urinary Incontinence treatment comprising one active ingredient wherein the at least one active ingredient is derived from a natural source, in combination with at least one other active ingredient; wherein the at least one other active ingredient is derived from a natural, artificial or semi-artificial source; and electively at least one excipient.

### BACKGROUND

The present invention relates to novel compositions for Urinary Incontinence treatment comprising at least one agent derived from a natural source as active agent, either alone or in combination with other active agent(s).

Herbal products have medically benefited nearly every culture on earth. Ancient people in Africa, Asia, Europe, and the Americas have practiced herbal medicine. The recent increase in the use of herbal products is associated with the belief that herbs can be utilized as herbal remedies and also in allopathic medicine to allow users to feel that they have some control in their choices of medications. In Africa particularly, use of herbal medicine is a long-standing tradition that offers a unique insight into approaches for the prevention and/or treatment of various human diseases.

Vernonia amygdaline plant, also known as rawa (Amharic), ewuro (Yoruba), etidot (Ibibio), onugbu (Igbo), ityuna (Tiv), oriwo (Edo), chusar-doki (Hausa), muluuza (Luganda), labwori (Acholi), and olusia (Luo) is cultivated in Africa and grows frequently in that region.

Presently no literature has been found relating to the use of Vernonia amygdaline for the treatment of Urinary Incontinence. Urinary incontinence is loss of bladder control due to age, prostate enlargement, or prostate cancer. Symptoms of said problem can range from mild leaking to uncontrollable wetting and it can happen to anyone, but it becomes more common with age.

Therefore, a need exists for a continued search for novel natural products that may be used for treatment of Urinary Incontinence through natural remedies. The present invention not only provides a novel solution to the aforementioned problem, also describes various compositions comprising an extract of Olugbu in combination with at least one other active ingredient for treatment of urinary incontinence.

### BRIEF SUMMARY OF INVENTION

This disclosure presents a composition for Urinary Incontinence treatment comprising at least one active ingredient; wherein the at least one active ingredient is derived from a natural source in combination with at least one other active ingredient; wherein the at least one other active ingredient is derived from a natural, artificial or semi-artificial source; and electively at least one excipient.

Furthermore, a process of making a composition for Urinary Incontinent treatment comprising steps of extraction from at least one active agent its essence; wherein the at least one active agent derived from a natural source; combination of at least one active agent with at least one other active agent; wherein the at least one other active agent is derived from a natural, artificial or semi-artificial source; and addition optionally at least one excipient.

Furthermore, a method of treating Urinary Incontinence comprising: administrating to an individual a composition comprising at least one active agent; wherein the at least one active agent is derived from a natural source in combination with at least one other active agent; wherein the at least one other active agent is derived from a natural, artificial or semi-artificial source; and electively at least one excipient.

### DETAILED DESCRIPTION AND EXAMPLES

Techniques described herein relate generally to various compositions and methods of manufacturing of a composition for treatment of Urinary Incontinence.

In present invention, a composition for treatment of Urinary Incontinence is produced. The composition may comprise of at least two active ingredients and electively at least one excipient. The active ingredients of the composition comprise of a first ingredient extractable from a natural source. The first ingredient may be the essence of Vernonia amygdaline Plant.

In present invention, the essence of Vernonia amygdaline plant may be extracted from its leaves, seeds, stems or a combination thereof. Further, various processes may be employed to extract the essence of the plant. For example, by placing the plant under pressure, the essence may be extracted.

Also, heating the plant to vaporize its internal fluid and subsequently capturing the evaporated fluid through a condensing process may be employed to extract the plant's internal fluid. Further, the extraction of the essence of Vernonia amygdaline plant may be carried out with a polar solvent applicable for extraction. Leaves are first extracted with a polar solvent optionally by several times. The obtained solution is then mixed and extracted with a non-polar solvent to remove the waxes, essential oils, pigments and most of the non-polar molecules.

In addition to aforementioned methods for capturing the essence of the plant, one may use parts of the plant to create the composition for treating Urinary Incontinence.

Second active ingredient may be obtained from a natural, artificial, or semi-artificial source. In one embodiment, the active ingredient is honey, wherein the honey can be procured from many sources including artificial, semi-artificial and natural sources. The honey may be mixed with the essence of the Vernonia amygdaline plant to produce the Urinary Incontinence Composition. Here, honey may be combined with the essence of Vernonia amygdaline plant (hereafter “Essence”).

In one embodiment of the present invention, the composition comprises one or more pharmaceutically acceptable excipients selected from but not limited to the group comprising diluents, disintegrants, binders, anti-adherants, glidants, anti-oxidants, buffering agents, colorants, flavoring agents, coating agents, solvents, viscosifying agents, waxes, wetting agents, emulsifying agents, solubilizers, stabilizers, buffering agents, vehicles, preservatives, surfactants, deodorants, colorants, and the like.

Moreover in present invention, honey and Essence may be having equal part weights in the composition. A range wherein one of the active ingredients has less than half part weight of the other active ingredient to where both active ingredients have equal part weights in the composition is considered. The effectiveness of the composition on an individual with Urinary Incontinence may vary by changing the ratio of Essence's part weight to honey's part weight in the composition. For example, the composition may comprise 20 percent Essence and 80 percent honey, in one composition may comprise 50 percent Essence and 50 honey, or one composition may comprise 70 percent Essence and 30 percent honey. As the percentage of Essence in the composition increases, the composition remedial effect may last longer in a subject.

It has been observed that the composition having equal Essence and honey part weights may treat the symptoms of Urinary Incontinence for four days in an individual with Urinary Incontinence. Further, when administrating the composition to a subject having Urinary Incontinence, wherein the Essence's part weight was half of the part weight of honey, the symptoms of Urinary Incontinence may be treated for two days. The composition may be administered to an individual with Urinary Incontinence in various forms including but not limit to liquid, cream, gel, lotion, spray, capsule or pill.

It is further the process of this invention to extract from at least one active ingredient its essence; wherein the at least one active ingredient derived from a natural source. Here the natural source is the Vernonia amygdaline plant. Furthermore, the process includes combining the essence of the Vernonia amygdaline plant with at least one other active ingredient; wherein the at least one other active ingredient is derived from a natural, artificial or semi-artificial source. The other active ingredient may be honey, wherein it can be produced from a natural, artificial or semi-artificial source.

The process may also include electively adding of at least one excipient. The excipient may be one of excipient selected from a group comprising diluents, disintegrants, binders, anti-adherants, glidants, lubricants, antioxidants, buffering ingredients, colorants, flavoring ingredients, coating ingredients, solvents, osmotic ingredients viscosifying ingredients, waxes, wetting ingredients, emulsifying ingredients, solubilizers, stabilizers, buffering ingredients, chelating ingredients, vehicles, preservatives, surfactants, deodorants, colorants, bulking ingredients, hydrophilic polymers, tonicity adjusting ingredients, local anesthetics, pH adjusting ingredients acids, sugar alcohol, reducing sugars and non-reducing sugars, or combination thereof.

In the aforementioned process, a range wherein one of the active ingredients has less than half part weight of the other active ingredient to where both active ingredients have equal part weights in the composition is considered.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the invention.

What is claimed is:

1. A composition for Urinary Incontinence treatment comprising:
at least one active ingredient; wherein the at least one active ingredient is derived from a natural source in combination with at least one other active ingredient;
wherein the at least one other active ingredient is derived from a natural, artificial or semi-artificial source; and
electively at least one excipient.

2. The composition of claim 1, wherein the at least one active ingredient is extracted from Vernonia amygdaline plant.

3. The composition of claim 1, wherein the at least one other active ingredient is honey.

4. The composition of claim 1, wherein the at least one other active ingredient is extracted from leaves Vernonia amygdaline plant.

5. The composition of claim 1, wherein the at least one active ingredient and the at least one other active ingredient have equivalent part weights in the composition.

6. The composition of claim 1, wherein the at least one active ingredient and the at least one other active ingredient have different part weights in the composition.

7. The composition of claim 1, wherein the composition is in the form of a topical preparation selected form a group comprising liquid, cream, gel, lotion, spray, capsule or pill.

8. The composition of claim 1, wherein the at least one excipient selected from a group comprising diluents, disintegrants, binders, anti-adherants, glidants, lubricants, antioxidants, buffering ingredients, colorants, flavoring ingredients, coating ingredients, solvents, osmotic ingredients viscosifying ingredients, waxes, wetting ingredients, emulsifying ingredients, solubilizers, stabilizers, buffering ingredients, chelating ingredients, vehicles, preservatives, surfactants, deodorants, colorants, bulking ingredients, hydrophilic polymers, tonicity adjusting ingredients, local anesthetics, pH adjusting ingredients acids, sugar alcohol, reducing sugars and non-reducing sugars, or combination thereof.

9. A process of making a composition for Urinary Incontinent treatment comprising steps of:
extraction essence of at least one active ingredient; wherein the at least one active ingredient derived from a natural source;
combination of the essence of at least one active ingredient with at least one other active ingredient; wherein the at least one other active ingredient is derived from a natural, artificial or semi-artificial source; and
electively addition of at least one excipient.

10. The process of claim 9, wherein the at least one active ingredient extracted from Vernonia amygdaline plant.

11. The process of claim 9, wherein the at least one other active ingredient is honey.

12. The process of claim 9, wherein the at least one excipient selected from a group comprising diluents, disintegrants, binders, anti-adherants, glidants, lubricants, antioxidants, buffering ingredients, colorants, flavoring ingredients, coating ingredients, solvents, osmotic ingredients viscosifying ingredients, waxes, wetting ingredients, emulsifying ingredients, solubilizers, stabilizers, buffering ingredients, chelating ingredients, vehicles, preservatives, surfactants, deodorants, colorants, bulking ingredients, hydrophilic polymers, tonicity adjusting ingredients, local anesthetics, pH adjusting ingredients acids, sugar alcohol, reducing sugars and non-reducing sugars, or mixtures thereof.

13. The process of claim 9, further comprising incorporation of the at least one active ingredient and the at least one other active ingredient in equivalent part weights in the composition.

14. The process of claim 9, further comprising incorporation of the at least one active ingredient and the at least one other active ingredient in dissimilar part weights in the composition.

15. A method of treating Urinary Incontinence comprising: administrating to an individual a composition comprising at least one active ingredient; wherein the at least one active ingredient is derived from a natural source in combination with at least one other active ingredient; wherein the at least one other active ingredient is derived from a natural, artificial or semi-artificial source; and electively at least one excipient.

16. The method of claim 15, wherein the at least one active ingredient is extracted from leaves of plant from Vernonia amygdaline plant, and the at least one other active ingredient is honey.
